(19) United States
(12) Patent Application Publication
(10) Pub. No.: US 2013/0023431 A1
(43) Pub. Date: Jan. 24, 2013

(54) Methods of Assessing Chromosomal Instabilities

(71) Applicant: Anthony P. Shuber, Mendon, MA (US)

(72) Inventor: Anthony P. Shuber, Mendon, MA (US)

(73) Assignee: PREDICTIVE BIOSCIENCES, INC., Lexington, MA (US)

(21) Appl. No.: 13/472,262

(22) Filed: May 15, 2012

Related U.S. Application Data
(63) Continuation-in-part of application No. 13/364,840, filed on Feb. 2, 2012.
(60) Provisional application No. 61/509,898, filed on Jul. 20, 2011.

(51) Int. Cl. G01N 33/574; C40B 30/04; C12Q 1/68

(52) U.S. Cl. 506/9; 435/6.14; 435/6.12; 436/501; 435/6.11

(57) ABSTRACT

Method of assessing or quantifying chromosomal instability in a subject, wherein the sample is obtained from an effluent, lavage, or organ wash. Methods of obtaining a whole genome sequence from an effluent, lavage, or organ wash.

### STATEMENT OF RELATED APPLICATIONS

This application is a continuation-in-part of U.S. patent application Ser. No. 13/364,840 filed Feb. 2, 2012, which claims the benefit of priority to U.S. Provisional Patent Application Ser. No. 61/509,898 filed on Jul. 20, 2011, the contents of both of which are hereby incorporated by reference in their entireties.

### FIELD OF THE INVENTION

The invention generally relates to methods for assessing chromosomal instabilities.

### BACKGROUND

A tissue biopsy is a standard technique employed by surgeons to obtain a sample of diseased tissue within a subject, such as a sample of a tumor within an organ. Generally, the biopsied sample is analyzed to determine the genetic make-up of the sample, allowing a doctor to diagnosis the type, grade, stage, and progression of a cancer afflicting the subject while also providing important insight into the best course of treatment for the subject.

While tissue biopsy has been used for many years to assess the genetic makeup of a tumor, recent studies suggest that there may be significant genetic heterogeneity within a tumor. These studies suggest that to more accurately determine the molecular genotype of a tumor, a sample is required that is representative of the genetic heterogeneity of the tumor. A tissue biopsy rarely provides this type of representative sample and therefore genetic analysis of biopsied tissue may not accurately reflect the molecular status of the whole tumor. This sampling error may have a great impact on the course of treatment for the subject that has undergone the tissue biopsy.

An alternative technique is to collect a body fluid from an organ of interest. This method is superior to a tissue biopsy in that a body fluid sample is typically more representative of the genetic makeup of the tumor. Additionally, it is often possible to identify molecular markers corresponding to the tumor in the body fluid. Nonetheless, the tumor specific nucleic acid is diluted significantly with non-tumor nucleic acid, making extraction and analysis difficult. For example, in a typical body fluid sample from an organ, altered nucleic acids containing mutations of interest are typically only present in small amounts (e.g., less than 1%) relative to a total amount of nucleic acids in the sample.

While it is possible to pull rare events out of the genetic data produced from bodily fluids, doing so takes a great amount of time and effort. Searching for rare events requires large data sets in order to build meaningful confidence levels. Such analyses are typically limited to identifying known abnormalities or defects, because of the existence of hybridization probes or primers specific to the suspected abnormality or defect. If the entire (or partial) genome of the rare event is sought, however, the problem becomes so complex as to be untenable. For example, Whole Genome Sequencing (WGS) or partial genome sequencing of a sample from a bodily fluid is very difficult using present technology without some other form of screening the sample for the rare event prior to analysis.

### SUMMARY

The invention generally relates to methods of assessing chromosomal instabilities in nucleic acid obtained from an organ wash. The organ wash is collected during an already existing standard-of-care diagnostic (e.g., colon wash during a colonoscopy or bladder washing during a cystoscopy). The resulting sample contains significantly more tumor enriched nucleic acid than a body fluid sample. Due to this tumor enriched nucleic acid sample, more complex analysis, such as whole genome sequencing, can be applied with little concern of getting results on nucleic acid not representative of an entire tumor.

Chromosome instability (CIN) describes, for example, an increased rate of chromosome missegregation during mitosis resulting in a failure to maintain the correct chromosomal arrangement (euploidy). Typically, an aberrant chromosomal state of a cell is classified based on the changes in arrangement of genetic material, gain or loss of whole chromosomes (aneuploidy) or gross chromosomal rearrangements (GCR), all of which are hallmarks of solid cancers. CIN can be caused by multiple mechanisms, including defects in mitotic spindle assembly, sister chromatid cohesion defects, increased microtubule attachments, or the presence of extra centrosomes. Research suggests that CIN occurs early in cancer development, and is often associated with poor prognosis.

In one embodiment, a method of the invention comprises providing a sample from an effluent, lavage, or organ wash of the subject, obtaining genetic material from the sample, and analyzing the genetic material for a chromosomal instability. In particular, the genetic material can be analyzed for genomically unstable loci, and a weighted value can be assigned to each genomically unstable locus, thereby quantifying the chromosomal instability. For example, a weighted value can be assigned based upon the severity, location, or type of chromosomal instability. In some instances, a weighted value can be assigned based upon location on a chromosome, proximity to telomeres, or proximity to known or suspected locations of genomic instabilities found in certain disorders.

In addition to identifying chromosomal instabilities, the methods of the invention can be used to track the progression or remission of a condition. In principle, the method would be repeated after a period in time, for example after a month, six months, a year, two years, five years, or ten years, and a new weighted value obtained. By comparing a prior and later weighted value, it is possible to determine if the condition or disorder has improved or worsened.

Using the methods of the invention it is possible to assess any effluent, lavage, or organ wash that has previously contacted an organ of the subject. For example the organ could be selected from the group consisting of lung, colon, bladder, cervix, vagina, kidney, spinal cord, brain, mouth, tongue, throat, and skin. The wash may be as simple as water, or the wash may additionally comprise stabilizers or preservatives to facilitate recovery of genetic material. In some instances the effluent, lavage, or organ wash will comprise whole cells. In some instances, the cells will be tumor cells. In some instances the effluent, lavage, or organ wash will comprise chromosomes or portions of a chromosome.

In addition, the present invention generally relates to methods of sequencing a whole genome of a subject. In one embodiment, the method comprises providing a sample from an effluent, lavage, or organ wash of the subject, obtaining a genome from the sample, and sequencing the genome. Upon sequencing the genome, the sequence can be compared to known biomarkers to identify loci of the genome that are indicative of a disorder or an abnormality. Alternatively, if a previous sequence of the subject is available, the acquired sequence can be compared to a newly-obtained sequence in order to identify changes in the sequence. In some instances, the changes are indicative of progression or recurrence of a disorder associated with changes in the sequence. Samples for whole genome sequencing can be obtained as discussed above, e.g., the effluent, lavage, or organ wash may have contacted an organ selected from the group consisting of lung, colon, bladder, cervix, vagina, kidney, spinal cord, brain, mouth, tongue, throat, and skin.

### DETAILED DESCRIPTION

While it is possible to pull rare events out of the genetic data produced from biopsy samples and bodily fluids, doing so takes a great amount of time and effort. This problem is overcome by collecting organ washes during an already existing standard of care diagnostic procedure (e.g. colon effluent during a colonoscopy, bladder washing during a cystoscopy, lung lavage for a suspicious lesion, Cerebral Spinal Fluid (CSF) for an identified brain tumor, a cervical wash performed during a standard cervical exam and pap smear). In certain aspects, methods of the invention involve obtaining nucleic acid from an organ wash of an organ of a subject, and analyzing the nucleic acid for a chromosomal instability. Methods of the invention can be performed with organ wash from any organ. Exemplary organs include liver, lung, colon, bladder, cervix, vagina, kidney, spinal cord, brain, mouth, tongue, and throat.

### Isolation of DNA

The methods of the invention may be carried out on purified or unpurified nucleic acid-containing samples. However, in particular embodiments, nucleic acid from organ wash is used. Any suitable nucleic acid isolation technique may be utilized. Examples of purification techniques may be found in standard texts such as Molecular Cloning—A Laboratory Manual (Third Edition), Sambrook and Russell (see in particular Appendix 8 and Chapter 5 therein). In one embodiment, purification involves alcohol precipitation of nucleic acid, such as DNA. Preferred alcohols include ethanol and isopropanol. Suitable purification techniques also include salt-based precipitation methods. Thus, in one specific embodiment the nucleic acid purification technique comprises use of a high concentration of salt to precipitate contaminants. The salt may comprise, consist essentially of or consist of potassium acetate and/or ammonium acetate for example. The method may further include steps of removal of contaminants which have been precipitated, followed by recovery of nucleic acid through alcohol precipitation.

In some embodiments, the effluent, lavage, or organ wash is concentrated before nucleic acid extraction. For example, the effluent, lavage, or organ wash may be filtered, centrifuged, settled, or extracted to concentrate the genetic material. In some embodiments, the effluent, lavage, or organ wash will contain cells which will be lysed in order to extract the genetic material. In some embodiments, organic solvents may be used to extract contaminants from the cell lysates. Thus, in one embodiment, the method comprises use of phenol, chloroform and isoamyl alcohol to extract the nucleic acid. Suitable conditions are employed to ensure that the contaminants are separated into the organic phase and that nucleic acid remains in the aqueous phase. In other embodiments, salts may be used to extract contaminants from the cell lysates.

In specific embodiments of these purification techniques, extracted nucleic acid is recovered through alcohol precipitation, such as ethanol or isopropanol precipitation. Amplification of nucleic acid from natural sources is often inhibited by co-purified contaminants and various methods adopted for nucleic acid extraction from environmental samples are available and provide an alternative for isolating nucleic acid from effluents, lavage, or organ wash according to specific embodiments of the invention. For instance, the QIAamp DNA Stool Mini Kit from QIAGEN adsorbs DNA-damaging substances and PCR inhibitors present in the sample by InhibitEX. Other examples for application in particular to faecal samples include the Wizard Genomic DNA Purification Kit (Promega), the NucliSENS® easyMAG™ (Biomerieux) and nucleic acid purification kits manufactured by Macherey Nagel.

In other embodiments, the DNA may be isolated by phenol-chloroform extraction since this has been shown to provide particularly high levels of DNA recovery from the sample.

Where blood is also in the effluent, lavage, or organ wash, the ChargeSwitch procedure may be utilized for example.

Suitable methods and kits for isolating nucleic acid from effluent, lavage, or organ wash are commercially available. Examples, each of which may be utilised in the methods of the invention are provided in the table below. Thus, as can be derived from Table 1, nucleic acid isolation may be carried out using silica-membranes, isopropanol or magnetic bead based methods for example.

TABLE 1
Kits and methods for isolating DNA from blood samples.

| Kit | Company | Method |
|---|---|---|
| UltraClean-htp ™ BloodSpin ™ | Mo Bio | Silica-membrane |
| DNA | Laboratories, | |
| | Inc. | |
| PAXgene Blood DNA Kit | Qiagen | isopropanol |
| QIAamp DNA Blood Maxi/Mini Kit | Qiagen | Silica-membrane |
| FlexiGene DNA Kit | Qiagen | isopropanol |
| GeneCatcher gDNA [symbol] -10 ml Blood | Invitrogen | magnetic beads |
| BC-204-10 ml-blood - Blood 10 ml | Baseclear | magnetic beads |
| ZR Genomic DNA I Kit | Zymo research | magnetic beads |
| DNAzol BD | MRC. Inc. | isopropanol |
| Gentra pureGene* DNA Purification | Fischer | isopropanol |
| Blood | | |
| MasterPure Whole Blood DNA | Epicentro | isopropanol |
| | Biotech. | |
| Invisorb ® Blood Giga Kit | Westburg | isopropanol |
| 100436-10 (Maxi) | Bioron | Silica-membrane |
| MagNA Pure LC DNA Isolation Kit | Roche | magnetic beads |
| Nuclisens EasyMag | Biomėneux | magnetic beads |
| chemagic blood kit special | chemagen | magnetic beads |

[symbol]

indicates data missing or illegible when filed

### Amplification of Epigenetic Elements

### Primers

Primers can be prepared by a variety of methods including but not limited to cloning of appropriate sequences and direct chemical synthesis using methods well known in the art (Narang et al., Methods Enzymol. 68:90 (1979); Brown et al., Methods Enzymol. 68:109 (1979)). Primers can also be obtained from commercial sources such as Operon Technologies, Amersham Pharmacia Biotech, Sigma, and Life Technologies. The primers can have an identical melting temperature. The lengths of the primers can be extended or shortened at the 5′ end or the 3′ end to produce primers with desired melting temperatures. Also, the annealing position of each primer pair can be designed such that the sequence and, length of the primer pairs yield the desired melting temperature. The simplest equation for determining the melting temperature of primers smaller than 25 base pairs is the Wallace Rule (Td=2(A+T)+4(G+C)). Computer programs can also be used to design primers, including but not limited to Array Designer Software (Arrayit Inc.), Oligonucleotide Probe Sequence Design Software for Genetic Analysis (Olympus Optical Co.), NetPrimer, and DNAsis from Hitachi Software Engineering. The Tₘ (melting or annealing temperature) of each primer is calculated using software programs such as Oligo Design, available from Invitrogen Corp.

The annealing temperature of the primers can be recalculated and increased after any cycle of amplification, including but not limited to cycle 1, 2, 3, 4, 5, cycles 6-10, cycles 10-15, cycles 15-20, cycles 20-25, cycles 25-30, cycles 30-35, or cycles 35-40. After the initial cycles of amplification, the 5′ half of the primers is incorporated into the products from each loci of interest, thus the Tₘ, can be recalculated based on both the sequences of the 5′ half and the 3′ half of each primer.

### Polymerase

Any RNA or DNA polymerase that catalyzes primer extension can be used including but not limited to E. coli DNA polymerase, Klenow fragment of E. coli DNA polymerase 1, T7 DNA polymerase, T4 DNA polymerase, Taq polymerase, Pfu DNA polymerase, Vent DNA polymerase, bacteriophage 29, REDTaq™, genomic DNA polymerase, sequenase, a recombinase, or a DNA helicase. In some embodiments, a thermostable DNA polymerase is used. A “hot start” PCR can also be performed wherein the reaction is heated to 95° C. for two minutes prior to addition of the polymerase or the polymerase can be kept inactive until the first heating step in cycle 1. “Hot start” PCR can be used to minimize nonspecific amplification. Any number of PCR cycles can be used to amplify the nucleic acid, including but not limited to 2, 5, 10, 15, 20, 25, 30, 35, 40, or 45 cycles.

### Amplification Methods

Any nucleic acid amplification-based technique may be used in accordance with the methods of the invention. For example, the epigenetic element of interest may be amplified using traditional PCR methods. In certain embodiments, the epigenetic element of interest is amplified using reverse transcriptase polymerase chain reaction (RT-PCR). RT-PCR is a well known technique in the art which relies upon the enzyme reverse transcriptase to reverse transcribe mRNA to form cDNA, which can then be amplified in a standard PCR reaction. Protocols and kits for carrying out RT-PCR are extremely well known to those of skill in the art and are commercially available.

Both PCR and RT-PCR can be carried out in a non-quantitative manner. End-point PCR and RT-PCR each measure changes in expression levels using three different methods: relative, competitive and comparative. These traditional methods are well known in the art. Alternatively, PCR or RT-PCR is carried out in a real time and/or in a quantitative manner. Real time quantitative PCR methods have been thoroughly described in the literature (see Gibson et al, Genome Res., 6(10):995-1001 (1996) for an early example of the technique) and a variety of techniques are possible. Examples include use of hydrolytic probes (Taqman™), hairpin probes (Molecular Beacons), FRET probe pairs (LightCycler™ (Roche)), hairpin probes attached to primers (Scorpion™), hairpin primers (Plexor™ and Amplifluor™), DzyNA and oligonucleotide blocker systems. All of these systems are commercially available and well characterized, and may allow multiplexing (that is, the determination of expression of multiple genes in a single sample).

Taqman™ was one of the earliest available real-time PCR techniques and relies upon a probe which binds between the upstream and downstream primer binding sites in a PCR reaction. A Taqman™ probe contains a 5′ fluorophore and a 3′ quencher moiety. Thus, when bound to its binding site on the DNA the probe does not fluoresce due to the presence of the quencher in close proximity to the fluorophore. During amplification, the 5′-3′ exonuclease activity of a suitable polymerase such as Taq digests the probe if it is bound to the strand being amplified. This digestion of the probe causes displacement of the fluorophore. Release of the fluorophore means that it is no longer in close proximity to the quencher moiety and this therefore allows the fluorophore to fluoresce. The resulting fluorescence may be measured and is in direct proportion to the amount of target sequence that is being amplified. These probes are sometimes generically referred to as hydrolytic probes.

In the Molecular Beacons system, the probe is again designed to bind between the primer binding sites. However, here the probe is a hairpin shaped probe. The hairpin in the probe when not bound to its target sequence means that a fluorophore attached to one end of the probe and a quencher attached to the other end of the probe are brought into close proximity and therefore internal quenching occurs. Only when the target sequence for the probe is formed during the PCR amplification does the probe unfold and bind to this sequence. The loop portion of the probe acts as the probe itself, while the stem is formed by complimentary arm sequences (to respective ends of which are attached the fluorophore and quencher moiety). When the beacon probe detects its target, it undergoes a conformational change forcing the stem apart and this separates the fluorophore and quencher. This causes the energy transfer to the quencher to be disrupted and therefore restores fluorescence.

During the denaturation step, the Molecular Beacons assume a random-coil configuration and fluoresce. As the temperature is lowered to allow annealing of the primers, stem hybrids form rapidly, preventing fluorescence. However, at the annealing temperature, Molecular Beacons also bind to the amplicons, undergo conformational reorganization, leading to fluorescence. When the temperature is raised to allow primer extension, the Molecular Beacons dissociate from their targets and do not interfere with polymerization. A new hybridization takes place in the annealing step of every cycle, and the intensity of the resulting fluorescence indicates the amount of accumulated amplicon.

Scorpion™ primers are based upon the same principles as Molecular Beacons. However, here, the probe is bound to, and forms an integral part of, an amplification primer. The probe has a blocking group at its 5′ end to prevent amplification through the probe sequence. After one round of amplification has been directed by this primer, the target sequence for the probe is produced and to this the probe binds. Thus, the name “scorpion” arises from the fact that the probe as part of an amplification product internally hybridizes to its target sequence thus forming a tail type structure. Probe-target binding is kinetically favoured over intrastrand secondary structures. Scorpion™ primers were first described in the paper “Detection of PCR products using self-probing amplicons and fluorescence” (Nature Biotechnology. 17, p 804-807 (1999)) and numerous variants on the basic theme have subsequently been produced.

In similar fashion to Scorpion™ primers, Amplifluor™ primers rely upon incorporation of a Molecular Beacon type probe into a primer. Again, the hairpin structure of the probe forms part of an amplification primer itself. However, in contrast to Scorpion™ type primers, there is no block at the 5′ end of the probe in order to prevent it being amplified and forming part of an amplification product. Accordingly, the primer binds to a template strand and directs synthesis of the complementary strand. The primer therefore becomes part of the amplification product in the first round of amplification. When the complimentary strand is synthesized amplification occurs through the hairpin structure. This separates the fluorophore and quencher molecules, thus leading to generation of florescence as amplification proceeds.

DzyNA primers incorporate the complementary/antisense sequence of a 10-23 nucleotide DNAzyme. During amplification, amplicons are produced that contain active (sense) copies of DNAzymes that cleave a reporter substrate included in the reaction mixture. The accumulation of amplicons during PCR/amplification can be monitored in real time by changes in fluorescence produced by separation of fluorophore and quencher dye molecules incorporated into opposite sides of a DNAzyme cleavage site within the reporter substrate. The DNAzyme and reporter substrate sequences can be generic and hence can be adapted for use with primer sets targeting various genes or transcripts (Todd et al., Clinical Chemistry 46:5, 625-630 (2000)).

The Plexor™ qPCR and qRT-PCR Systems take advantage of the specific interaction between two modified nucleotides to achieve quantitative PCR analysis. One of the PCR primers contains a fluorescent label adjacent to an iso-dC residue at the 5′ terminus. The second PCR primer is unlabeled. The reaction mix includes deoxynucleotides and iso-dGTP modified with the quencher dabcyl. Dabcyl-iso-dGTP is preferentially incorporated at the position complementary to the iso-dC residue. The incorporation of the dabcyl-iso-dGTP at this position results in quenching of the fluorescent dye on the complementary strand and a reduction in fluorescence, which allows quantitation during amplification. For these multiplex reactions, a primer pair with a different fluorophore is used for each target sequence.

Real time quantitative techniques for use in the invention generally produce a fluorescent read-out that can be continuously monitored. Fluorescence signals are generated by dyes that are specific to double stranded DNA, like SYBR Green, or by sequence-specific fluorescently-labeled oligonucleotide primers or probes. Each of the primers or probes can be labeled with a different fluorophore to allow specific detection. These real time quantitative techniques are advantageous because they keep the reaction in a “single tube”. This means there is no need for downstream analysis in order to obtain results, leading to more rapidly obtained results. Furthermore, keeping the reaction in a “single tube” environment reduces the risk of cross contamination and allows a quantitative output from the methods of the invention. This may be particularly important in a clinical setting for the present invention.

In some embodiments, the labeled probe comprises a reporter molecule at the 5′ end, a quencher molecule at the 3′ end. In certain embodiments, the reporter molecule at the 5′ end of the chimeric probe is a fluorophore, such as 6-carboxyfluoroscein (FAM), tetrachlorofluoroscein (TET), HEX, TAMRA, ROX, CY3, CY3.5, Texas Red, CY5, CY5.5, CY7, or an Alexa dye, and the quencher molecule at the 3′ end of the probe is tetramethylrhodamine (TAMRA) or dihydrocyclopyrroloindole tripeptide minor groove binder (MGB). In particular embodiments, said probe is a TAQMAN probe, a molecular beacon, a Scorpion™ primer/probe, a FRET probe, a Light Cycler™ probe, or an Amplifluor™ primer/probe. Probes for use in real-time PCR techniques, such as TAQMAN probes, molecular beacons, Scorpion™ primers/probes, and FRET probes can be custom designed using commercially available software such as Beacon Designer™ (Premier Biosoft International, Palo Alto, Calif.). Probes for use in real-time PCR can also be custom ordered through commercial sources (see, for example, custom TAQMAN probe services offered by Applied Biosystems; custom molecular beacon, Scorpion™ and Light Cycler™ probe services offered by Sigma-Aldrich, St. Louis, Mo.; and custom Amplifluor™ primer services offered by Biosearch Technologies, Novato, Calif.).

The use of probes in conjunction with primers enables highly sensitive qPCR reactions. Chimeric primers have a Tₘ that is approximately 12°-18° C. higher than standard primers (e.g., Tₘ of 72° C. vs. Tₘ of 56° C., respectively). Likewise, probes have a higher Tₘ than that of standard probes (e.g., Tₘ 80° C. vs. Tₘ 68° C., respectively). The higher Tₘ allows for the annealing temperature to be increased thereby decreasing mismatch tolerance and increasing assay sensitivity and specificity.

It should be noted that while PCR is a preferred amplification method, to include variants on the basic technique such as nested PCR, equivalents may also be included within the scope of the invention. Examples include without limitation isothermal amplification techniques such as NASBA, 3SR, TMA and triamplification, all of which are well known in the art and commercially available. Other suitable amplification methods without limitation include the ligase chain reaction (LCR) (Barringer et al, Proc Natl Acad Sci, 87(5):1874-1878 (1990)) MLPA, selective amplification of target polynucleotide sequences (U.S. Pat. No. 6,410,276), consensus sequence primed polymerase chain reaction (U.S. Pat. No. 4,437,975), invader technology (Third Wave Technologies, Madison, Wis.), strand displacement technology, arbitrarily primed polymerase chain reaction (WO90/06995) and nick displacement amplification (WO2004/067726).

In some embodiments, the amplification method used in accordance with the methods of the invention is recombinase polymerase amplification (RPA). RPA is an isothermal reaction that utilizes recombinases which are capable of pairing oligonucleotide primers with homologous sequence in duplex DNA. Through this method, DNA synthesis is directed to defined points in a sample DNA. If the target sequence is indeed present, DNA amplification reaction is initiated; no other sample manipulation such as thermal or chemical melting is required. The reaction progresses rapidly and results in specific DNA amplification from just a few target copies to detectable levels typically within 5-10 minutes. The entire reaction system is stable as a dried formulation and can be transported safely without refrigeration.

Specific benefits of the RPA technology include a number of features which make it suited for portable and point-of-use applications. For example, RPA operates at low, constant temperatures and does not require initial melting of the sample DNA (optimal temperature 37° C.); in fact, body heat can support the process if necessary. It is also fully robust in the face of off-temperature and low temperature set-up. At typical ambient temperatures (25° C.) the process works, albeit more slowly, and results can still be obtained within an hour with the biochemistry appropriately configured. RPA has been shown to function directly on crude samples from a number of sources, including blood samples and nasal swabs as well as culture media, without any requirement for nucleic acid purification (using simple lysis methods such as weak alkali or heat lysis). This extraordinary robustness to complex samples makes RPA ideally suitable for field use and point-of-care applications. Additionally, RPA can detect single copies of DNA and tens of copies of RNA (or less). RPA is also so specific that it can operate easily to single molecule levels in the presence of hundreds of nanograms of unrelated complex genomic DNA, such as that from plants and mammals. This permits the detection of trace levels of targets even in extremely complex nucleic acid samples. As such, RPA is particularly suitable for use in the amplification and detection of epigenetic elements, which are typically present in very limited amounts in a given biological sample. See products offered by TwistDx Limited (Cambridge, UK).

In some embodiments, helicase-dependent amplification (HDA) may be used in accordance with the methods of the invention. Like RPA, HDA is an isothermal reaction that utilizes a DNA helicase to generate single-stranded templates for primer hybridization. Subsequent primer extension is then catalyzed by a DNA polymerase. HDA does not require an expensive thermocycler and thus amplification of nucleic acid may be performed practically anywhere. In addition, it offers several advantages over other isothermal DNA amplification methods by having a simple reaction scheme and being a true isothermal reaction that can be performed at one temperature for the entire process. See Vincent M, Xu Y, Kong H. Helicase-dependent isothermal DNA amplification. EMBO Rep. 2004 August; 5(8):795-800. Epub 2004 Jul. 9; See also products offered by Biohelix Corp. (Beverly, Mass.).

In certain embodiments, methylation-specific PCR (MSP) is used in accordance with the methods of the invention. MSP is used to detect methylation of CpG islands in genomic DNA. DNA is first treated with sodium bisulfite, which converts unmethylated cytosine bases to uracil, which is recognized by PCR primers as thymine. Two PCRs are then carried out on the modified DNA, using primer sets identical except at any CpG islands within the primer sequences. At these points, one primer set recognizes DNA with cytosines to amplify methylated DNA, and one set recognizes DNA with uracil or thymine to amplify unmethylated DNA. See EpiTect Bisulfite Kits and PCR reagents offered by QIAGEN (Valencia, Calif.). See also, Herman et al., “Methylation-specific PCR: A novel PCR assay for methylation status of CpG islands,” P.N.A.S., vol. 93, p. 9821-26 (1996), which is incorporated herein by reference in its entirety. This technique can detect methylation changes as small as ±0.1%. In addition to methylation of CpG islands, many of the sequences surrounding clinically relevant hypermethylated CpG islands can also be hypermethylated, and are potential biomarkers.

Preferably, the MSP is real-time quantitative MSP (QMSP) which permits reliable quantification of methylated DNA. The QMSP method is based on the continuous optical monitoring of a fluorogenic PCR. This PCR approach can detect aberrant methylation patterns in human samples with substantial (1:10.000) contamination of normal DNA. See Eads et al., Nucl Acids Res 28(8):E32 (2000). Moreover, QMSP is amenable to high-throughput techniques allowing the analysis of close to 400 samples in less then 2 hours without requirement for gel-electrophoresis.

Beyond MSP, it is also possible to measure methylation levels by using hybridization probes that are specific for the products of bisulfate-converted nucleic acids using real-time PCR with primers that not complimentary to the CpG island regions of interest, or primers that hybridize to sequences adjacent to the CpG islands. Methods of using primers having abasic and or mismatch regions corresponding to CpG islands are disclosed in U.S. patent application Ser. No. 13/472,209 “Primers for Analyzing Methylated Sequences and Methods of Use Thereof,” filed May 15, 2012, and incorporated by reference herein in its entirety. Additionally, it is possible to determine an amount of methylation by amplifying and directly sequencing nucleic acids by using single molecule sequencing.

Other nucleic acid amplification techniques may also be modified to detect the methylation status of the panel of genes. Such amplification techniques are well known in the art, and include methods such as NASBA (see Compton, Nature 7; 350(6313): p 91-92 (1991)), 3SR (see Fahy et al., PCR Methods Appl. 1(1): p 25-33 (1991)) and Transcription Mediated Amplification (TMA). Amplification is achieved with the use of primers specific for the sequence of the gene whose methylation status is to be assessed. In order to provide specificity for the nucleic acid molecules primer binding sites corresponding to a suitable region of the sequence may be selected. The skilled reader will appreciate that the nucleic acid molecules may also include sequences other than primer binding sites which are required for detection of the methylation status of the gene, for example RNA Polymerase binding sites or promoter sequences may be required for isothermal amplification technologies, such as NASBA, 3SR and TMA.

TMA (Gen-probe Inc.) is an RNA transcription amplification system using two enzymes to drive the reaction, namely RNA polymerase and reverse transcriptase. The TMA reaction is isothermal and can amplify either DNA or RNA to produce RNA amplified end products. TMA may be combined with Gen-probe's Hybridization Protection Assay (HPA) detection technique to allow detection of products in a single tube. Such single tube detection is a preferred method for carrying out the invention.

The methylation status of a specific promoter of interest can also be analyzed using PCR techniques based on MeCP2 binding, which differentiates those promoters with methylated groups from unmethylated promoters. Isolated genomic DNA is digested with MseI, and the resulting DNA fragments are incubated with the methylation binding protein MeCP2 (a.k.a. MBP). The methylated DNA fragments are isolated with a spin column and the amplified with promoter specific primers. Agarose gel electrophoresis is used to visualize the PCR products. The presence of a band on the gel indicates that a specific promoter is methylated in the genomic DNA sample. See Methylation Promoter PCR Kits offered by Panomics/Affymetrix, Inc. (Santa Clara, Calif.).

### Detection of Epigenetic Elements

Amplicons of the epigenetic elements of interest can be detected using a variety of means known in the art. For example, oligonucleotide probes comprising sequence specific for epigenetic element of interest can be used, such as the various probes for use in real-time quantitative PCR, as previously described. Other suitable techniques for detecting nucleic acids are well known in the art and include, for example and not by way of limitation, northern or southern blotting, mass spectrometry and use of microarrays. Accordingly use of these well known techniques may be incorporated in the methods of the invention.

Techniques for detecting protein expression, such as prion levels include, but are not limited to, Immuno detection methods which can be broadly split into two main categories; solution-based techniques such as enzyme-linked immunosorbent assays (ELISA), immunoprecipitation and immunodiffusion, and procedures such as Western blotting and dot blotting where the samples have been immobilized on a solid support. Said methods rely on antibodies which recognize specifically the protein of interest. Said methods may be included in the methods of the present invention.

Aptamers may also be used to detect the epigenetic element of interest. Aptamers are small single stranded RNAs or DNAs approximately 40-100 base pairs in length that form secondary and tertiary structures which bind to other biological molecules with very high affinity and specificity. Optionally, the aptamer is labeled with an optically detectable species.

As stated herein, the methods are useful for detecting the methylation status of at least one gene. By “detecting the methylation status” is meant determining the presence or absence of 5-methylcytosine at one or a plurality of (functionally relevant) CpG dinucleotides within the DNA sequence of the at least one gene. In particular, aberrant methylation, which may be referred to as hypermethylation, of the at least one gene may be detected. Typically, the methylation status is determined in one or more CpG islands in the at least one gene. These CpG islands are often found in the promoter region of the gene(s). Thus, CpG dinucleotides are typically concentrated in the promoter regions and exons of human genes and the methylation status of these CpG residues is of functional importance to whether the at least one gene is expressed. Since CpG dinucleotides susceptible to methylation are typically concentrated in the promoter region, exons and introns of human genes, promoter, exon and intron regionsmay be assessed in order to determine the methylation status of the at least one gene. A “promoter” is a region extending typically between approximately 1 Kb, 500 by or 150 to 300 by upstream from the transcription start site. The CpG island may surround or be positioned around the transcription start site of the at least one gene.

Methods for detecting methylation status rely upon a reagent which selectively modifies unmethylated cytosine residues in the DNA contained in the sample to produce detectable modified residues but which does not modify methylated cytosine residues. Any suitable reagent may be utilized in the methods of the invention. Examples include bisulphite, hydrogen sulphite and disulphite reagents and suitable mixtures thereof. In an embodiment of the invention, the reagent comprises, consists essentially of or consists of a bisulphite reagent. In particular, the reagent may comprise, consist essentially of or consist of sodium bisulphite.

Methylation-sensitive restriction endonucleases can also be used to detect methylated CpG dinucleotide motifs. Such endonucleases may either preferentially cleave methylated recognition sites relative to non-methylated recognition sites or preferentially cleave non-methylated relative to methylated recognition sites. Examples of the former are Acc III, Ban I, BstN I, Msp I, and Xma I. Examples of the latter are Acc II, Ava I, BssH II, BstU I, Hpa II, and Not I. Alternatively, chemical reagents can be used which selectively modify either the methylated or non-methylated form of CpG dinucleotide motifs.

### Sequencing Epigenetic Elements

The methods of the invention may also, as appropriate, incorporate sequencing as the means for detecting the epigenetic element of interest after amplification. Alternatively, the methods of the invention may incorporate sequencing of the epigenetic element of interest before amplification or after amplification and detection by other means.

A variety of sequencing methods are well known in the art and can be incorporated into the methods of the invention. For example, classical chain termination sequencing methods can be used. The classical chain-termination method requires a single-stranded DNA template, a DNA primer, a DNA polymerase, radioactively or fluorescently labeled nucleotides, and modified nucleotides that terminate DNA strand elongation. The DNA sample is divided into four separate sequencing reactions, containing all four of the standard deoxynucleotides (dATP, dGTP, dCTP and dTTP) and the DNA polymerase. To each reaction is added only one of the four dideoxynucleotides (ddATP, ddGTP, ddCTP, or ddTTP) which are the chain-terminating nucleotides, lacking a 3′-OH group required for the formation of a phosphodiester bond between two nucleotides, thus terminating DNA strand extension and resulting in DNA fragments of varying length. The newly synthesized and labeled DNA fragments are heat denatured, and separated by size (with a resolution of just one nucleotide) by gel electrophoresis on a denaturing polyacrylamide-urea gel with each of the four reactions run in one of four individual lanes (lanes A, T, G, C); the DNA bands are then visualized by autoradiography or UV light, and the DNA sequence can be directly read off the X-ray film or gel image.

Technical variations of chain-termination sequencing, include tagging with nucleotides containing radioactive phosphorus for radiolabelling, or using a primer labeled at the 5′ end with a fluorescent dye (i.e., dye-primer sequencing) can also be used.

Dye-terminator sequencing can also incorporated into the methods of the invention to sequence the epigenetic element of interest before or after amplification. Dye-terminator sequencing utilizes labeling of the chain terminator ddNTPs, which permits sequencing in a single reaction, rather than four reactions as in the labeled-primer method. In dye-terminator sequencing, each of the four dideoxynucleotide chain terminators is labeled with fluorescent dyes, each of which emit light at different wavelengths. Protocols, reagents and equipment for carrying out chain-termination sequencing, dye-primer sequencing and dye-terminator sequencing are extremely well known to those of skill in the art and are commercially available.

In a particular embodiment, next-generation sequencing methods are incorporated into the methods of the invention to sequence the epigenetic element of interest before or after amplification. As described Braslaysky et al., “Sequence information can be obtained from single DNA molecules”, Proc. Nat. Acad. Sci. 100(7): 3960-3964 (2003), DNA polymerase may be employed to image sequence information in a single DNA template as its complementary strand is synthesized. The nucleotides are inserted sequentially; only the time resolution to discriminate successive incorporations is required. After each successful incorporation event, a fluorescent signal is measured and then nulled by photobleaching.

Briefly, this technique permits observations of single molecule fluorescence by a conventional microscope equipped with total internal reflection illumination, which reduces background fluorescence. The surface of a quartz slide is chemically treated to specifically anchor DNA templates while preventing nonspecific binding of free nucleotides, and a plastic flow cell is attached to the surface to exchange solutions. DNA template oligonucleotides are hybridized to a fluorescently labeled primer and bound to the surface via streptavidin and biotin with a surface density low enough to resolve single molecules. Polymerase and one fluorescently labeled nucleotide (C, G, A or T) are added. The polymerase catalyzes the sequence-specific incorporation of fluorescent nucleotides into nascent complementary strands on all the templates. After a wash step, which removes all free nucleotides, the incorporated nucleotides are imaged and their positions recorded. The fluorescent group is removed in a highly efficient cleavage process, leaving behind the incorporated nucleotide. The process continues through each of the other three bases. Multiple four-base cycles result in complementary strands greater than 25 bases in length synthesized on billions of templates—providing a greater than 25-base read from each of those individual templates. The technique uses a combination of evanescent wave microscopy and single-pair fluorescence resonance energy transfer (spFRET) to reject unwanted noise. The donor fluorophore excites acceptors only within the Forster radius, thus effectively creating an extremely high-resolution near-field source. Because the Forster radius of this fluorophore pair is 5 nm, the spatial resolution of this method exceeds the diffraction limit by a factor of 50 and conventional near-field microscopy by an order of magnitude. See products offered by Helicos, Inc. (Cambridge, Mass.), e.g., the HeliScope™ Single Molecule Sequencer. See Also products offered by Pacific Biosciences (California) e.g., the PacBio RS SMRT™ Sequencer.

Another methodology useful in the present invention platform is based on massively parallel sequencing of millions of fragments using attachment of randomly fragmented genomic DNA to a planar, optically transparent surface and solid phase amplification to create a high density sequencing flow cell with millions of clusters, each containing about 1,000 copies of template per sq. cm. These templates are sequenced using four-color DNA sequencing-by-synthesis technology. See, products offered by Illumina, Inc. (San Diego, Calif.) e.g., the HiSEQ2000 system. Also, see US 2003/0022207 to Balasubramanian, et al., published Jan. 30, 2003, entitled “Arrayed polynucleotides and their use in genome analysis.”

In particular embodiments of the methods of the invention, sequencing methods are used to detect and/or analyze methylated DNA. As previously described, bisulphite conversion of unmethylated cytosine is an easy and widely accepted method for detecting methylated CpGs. The gold standard technique for methylation detection is the sequencing of bisulphite treated DNA using automated capillary electrophoresis instruments. See, e.g., sequencing services offered by SeqWright DNA Technology Services (Houston, Tex.). High throughput sequencing methods and commercial kits for analyzing DNA methylation are also known. See e.g., The SOLiD™ System from Applied Biosciences (Foster City, Calif.).

Other high-throughput sequencing platforms are currently available. The Genome Sequencers from Roche/454 Life Sciences (Margulies et al. (2005) Nature, 437:376-380; U.S. Pat. Nos. 6,274,320; 6,258,568; 6,210,891) utilizes a micro plate, bead based format and luminescent detection from an enzyme cascade employing luciferase. The 1G Analyzer from Illumina/Solexa (Bennett et al. (2005) Pharmacogenomics, 6:373-382) utilizes amplified nucleic acid colonies on an arrayed planar surface chemistry and fluorescence detection. The SOLiD system from Applied Biosystems (solid.appliedbiosystems.com), and the HeliScope™ system from Helicos Biosciences (see, e.g., U.S. Patent App. Pub. No. 2007/0070349) utilizes arrayed single nucleic acid molecules on a planar surface and fluorescence detection. Although these new technologies are significantly cheaper compared to the traditional methods, such as gel/capillary Gilbert-Sanger sequencing, the sequence reads produced by the new technologies are generally much shorter (about 25-40 vs. about 500-700 bases). For example, the average read lengths on the four major platforms are currently as follows: Roche/454, 250 bases (depending on the organism); Illumina/Solexa, 25 bases; SoliD, 35 bases; Heliscope, 25 bases.

In certain embodiments, the sequencing method is a single molecule sequencing by synthesis method. Single molecule sequencing is shown for example in Lapidus et al. (U.S. Pat. No. 7,169,560), Lapidus et al. (U.S. Pat. No. 7,897,345), Quake et al. (U.S. Pat. No. 6,818,395), Harris (U.S. Pat. No. 7,282,337), Quake et al. (U.S. Pat. No. 7,297,518), and Braslaysky, et al., PNAS (USA), 100: 3960-3964 (2003), the contents of each of these references is incorporated by reference herein in its entirety.

### Quantification of Epigenetic Elements

The methods of the invention may also, as appropriate, incorporate quantification of the epigenetic element of interest before and/or after amplification. Quantification of the epigenetic element of interest may be achieved using any suitable means. For example, simultaneous amplification, detection and quantification of epigenetic elements may be accomplished according to the methods of the invention using qPCR, as previously described. Quantification of the epigenetic element of interest may also be based, for example, upon use of a spectrophotometer, a fluorometer or a UV transilluminator. Examples of suitable techniques are described in standard texts such as Molecular Cloning—A Laboratory Manual (Third Edition), Sambrook and Russell (see in particular Appendix 8 therein). In one embodiment, kits such as the Picogreen® dsDNA quantitation kit available from Molecular Probes, Invitrogen may be employed to quantitate the epigenetic element.

In the embodiments where the epigenetic element being amplified and detected according to the methods of the invention is methylated DNA, the methylated DNA can be quanitified using the quantitative methylation specific PCR (QMSP) methods, as previously described. Other suitable methods for quantifying methylated DNA include the measurement of levels of 5-methylcytosine (5-mC) in the amplified reaction mixture. For example, 5-methylcytosine (5-mC) can be quantified by binding the amplified DNA to a multiwall plate where the wells are specifically treated to have a high DNA affinity. The methylated fraction of DNA is detected using capture and detection antibodies and then quantified colorimetrically by reading the absorbance in a microplate spectrophotometer at 450 nm. The amount of methylated DNA is proportional to the OD intensity measured, which can be calculated using formulas for relative methylation status of two different DNA samples or absolute quantification of 5-methylcytosine (5-mC) using a standard curve. See e.g., the MethylFlash™ Methylated DNA Quantification Kit and the MethylFlash™ Hydroxymethylated DNA Quantification Kit from Epigentek (Brooklyn, N.Y.).

Another means for quantifying methylated DNA is Bio-COBRA (combined bisulfite restriction analysis coupled with the Agilent 2100 Bioanalyzer platform). The combination of a COBRA, which interrogates DNA methylation via the restriction enzyme analysis of PCR-amplified bisulfite treated DNAs, with the Agilgent Bioanalyzer platform allows for the rapid and quantitative assessment of DNA methylation patterns in large sample sets. See Brena et al., Nucl. Acids Res. 34(3):e17 (2006).

### Identifying Genomic Instabilities

All cancers are associated with some form of genomic instability. Abnormalities can range from a discrete mutation in a single base in the DNA of a single gene to a gross chromosome abnormality involving the addition or subtraction of an entire chromosome or set of chromosomes. A primary effort in cancer genetics is directed towards identifying specific genomic abnormalities associated with cancer. An exact match to a known genomic instability in cancer is often used for prognosis. Focusing only on genomic instabilities known to be associated with cancer, however, overlooks the reality that genomic instabilities found in patients often vary from the specific known genomic abnormalities found in cancer. Furthermore, many genomic instabilities not associated with the known cancer instabilities are not utilized in determining the grade, stage, and prognosis for the patient.

In some embodiments, methods of the invention are used to recover sample nucleic acid which is sequenced and compared to a reference sequence to determine the number of genomically unstable loci in the sample. The number of genomically unstable loci is then utilized in determining the grade, stage, and prognosis of the cancer for the patient. A biopsy sample is obtained and the nucleic acid is purified and sequenced. Either whole tumor genome sequencing or targeted tumor gene resequencing is performed in order to obtain the sequence. The sample is compared to a reference sequence, such as a human consensus sequence or a non-cancerous sample from the patient, such as a buccal swab. The number of genomically unstable loci is assessed compared to the reference sequence so that the grade, stage, and prognosis of the cancer are determined based upon the quantity of genomic instabilities.

Methods of the invention further provide for integrating the number of genomically unstable loci and a pathology report of the sample to comprehensively determine the stage, grade, and prognosis of cancer in the patient. Samples are obtained over time to determine a change in the number of genomically unstable loci present in the sample. The number of genomically unstable loci determined over time is compared to a baseline number of genomically unstable loci in order to assess tumor progression, response to treatment, etc.

Any genomically unstable loci are indicative of cancer. Generally, the greater the number of genomically unstable loci the more severe the cancer is, both in terms of prognosis and stage of the cancer.

Methods of the invention further provide for assessing cancer in a patient by obtaining a sample from an effluent, lavage, or organ wash and determining a number of genomically unstable loci in the sample. With the same sample, the number of genomically stable loci is also determined and the two numbers are compared to calculate a rational number. A rational number is any number that can be expressed as the quotient or fraction a/b of two integers. The number of genomically stable loci is divided by the number of genomically unstable loci to determine the rational number. The rational number is used to assess the stage, grade, and prognosis of the cancer. The number of genomically stable and unstable loci is determined by whole genome sequencing, targeted gene resequencing, PCR, DNA microarray, fluorescent in situ hybridization, Southern blot analysis, or Northern blot analysis.

Methods of this aspect of the invention also provide for integrating the rational number obtained with a pathology report of the sample to comprehensively determine the stage, grade, and prognosis of cancer in the patient. Over time a subsequent rational number is obtained from a second biopsy sample. Any change in the rational number at a subsequent time point compared to the baseline is a change in the stage, grade, and prognosis of cancer in the patient.

Methods of the invention further provide for assessing the efficacy of a therapeutic treatment for cancer by obtaining a first number of genomically unstable loci from a first effluent, lavage, or organ wash, and administering a therapeutic treatment to the patient. After the therapeutic treatment is administered, a second number of genomically unstable loci from a second effluent, lavage, or organ wash are assessed. The difference in the first number of genomically unstable loci compared to the second number of genomically unstable loci is indicative of determining the efficacy of the therapeutic treatment. If the difference between the first and second number of genomically unstable loci is decreased, the treatment is effective while if there is an increase or no change then that therapeutic treatment is ineffective and an alternate therapeutic treatment should be considered.

In other embodiments, all genomic instabilities within the effluent, lavage, or organ wash sample are quantitated. All genomic instabilities within a sample are not the same, and therefore assessing each genomic instability based on its own unique characteristics adds another level of information for in-depth prognosis. Genomic instabilities within a nucleic acid range in significance and severity in causing cancer. Embodiments of the invention provide for assigning a weighted value to each genomically unstable locus in the sample sequence in order to causally relate all genomic instabilities within the sample sequence to a cancer. The weighted value may be scaled in any manner, including and not limited to assigning a positive or negative integer to reflect the significance or severity of the locus as compared to a certain cancer sequence. The weighed value provides significant insight into the prognosis of cancer because each genomically unstable locus may be factored into determining the grade and stage of cancer, instead of only exact matches to known instabilities found in cancer. In one embodiment, a comparison of the weighted values over time and over courses of treatment allows one to alter treatment based on the specific variations of all instabilities linked to cancer.

In another embodiment an effluent, lavage, or organ wash sample is used to assess the stage and the grade of the cancer utilizing weighted values. After the method is performed in a first sample, the weighted values, sums, or averages are entered into a reference log. The method is then performed again on a second sample from the patient after a lapse in time or course of treatment. The weighted values, sums, or averages of the second sample are also entered into the reference log, and then compared to the first sample. Variances between the first sample's weighted values, sums, or averages and the second sample's weighted values, sums, or averages represents a change in stage or grade of the cancer. Therapeutic treatment may be tailored to the weighted variances providing a specialized treatment based on specific genomic instabilities.

### Determining Presence and Type of Genomically Unstable Loci

As described above, look up tables can be used to compare sequencing results to determine genomically unstable loci of the sequence. Once a genomic sequence from one sample has been determined by sequencing, as described above, hybridization techniques are used to determine variations in sequence between the sample sequence and a reference sequence. The variations between the two sequences are the genomically unstable loci of interest.

The number of genomically unstable loci are quantified for the sample and compared to that of a reference sequence in order to determine stage, grade, and prognosis of cancer in the patient. An example of a suitable hybridization technique involves the use of DNA chips (oligonucleotide arrays), for example, those available from Affymetrix, Inc. Santa Clara, Calif. Reference sequences for use in comparison to the sample sequence include, but are not limited to, a sample from a non-cancerous tissue taken from the subject, such as a buccal swab, or a human consensus sequence.

In other embodiments of the invention, a primer with predetermined genomically unstable loci that binds to the nucleic acid of the sample is indicative of that genomically unstable locus. The presence of specific genomically unstable loci in particular cancers can also determine the grade, stage, and prognosis of the cancer. One method of determining the presence of predetermined genomically unstable loci includes PCR. Methods for implementing PCR are well-known. In the present invention, human DNA fragments are amplified using human-specific primers. Amplicon of greater than about 200 bp produced by PCR represents a positive screen. Other amplification reactions and modifications of PCR, such as ligase chain reaction, reverse-phase PCR, Q-PCR, and others may be used to produce detectable levels of amplicon. Amplicon may be detected by coupling to a reporter (e.g. fluorescence, radioisotopes, and the like), by sequencing, by gel electrophoresis, by mass spectrometry, or by any other means known in the art, as long as the length, weight, or other characteristic of the amplicons identifies them by size.

### Quantitative Assessment of Genomically Unstable Loci

In certain embodiments of the invention, the number of genomically unstable loci is assessed over time. Methods as described above are performed on a second sample obtained from the same subject. The number of genomically unstable loci in the second sample are compared to that of the first to determine stage, grade, and prognosis of cancer in the patient.

In another aspect of the invention, a rational number is determined to assess the stage, grade, and prognosis of cancer in the patient. A rational number is any number that can be expressed as the quotient or fraction a/b of two integers. In this aspect, the number of genomically unstable loci is determined in a sample using methods described above. Further, the number of genomically stable loci from the same sample is determined and a ratio of the number of genomically unstable loci to genomically stable loci provides a rational number for that patient. As discussed above, any genomic instability is indicative of cancer. There is a linear relationship between the rational number and the severity of cancer in the patient, the higher the number, the worse the grade, stage, and prognosis of cancer.

In one embodiment of the invention, a rational number is obtained from the same sample of the subject over time. Any increase in the rational number of genomic instabilities, the more severe the cancer. A rational number that is progressing upwards over time indicates an increasing severity of cancer and ineffectiveness of the current therapeutic treatment. Any decrease in the rational number of genomically unstable loci to genomically stable loci is indicative of the improvement of the grade, stage, and prognosis of cancer for the patient from the previous analysis. Further, the rate at which the rational number changes indicates the severity of the cancer. The more severe the cancer, the steeper the slope will be between the rational number of at least two time points.

Based upon the determination of the number of genomically unstable loci and/or the presence of predetermined genomically unstable loci, methods of the invention also include providing targeted therapeutic treatment based upon the presence and/or quantity of genomically unstable loci in a sample.

### Providing and Recording Targeted Therapeutic Treatment Based on Quantitative Assessment

Methods of this invention are useful because the size of the tumor may shrink over the course of treatment while the tumor cells may remain as genomically unstable, if not more so, than when the tumor was a larger size. Alternatively, a tumor may remain the same size in an individual, while the number of genomically unstable loci may decrease, thus decreasing the stage, grade, and prognosis of cancer in the patient. Therefore, by assessing the presence of specific genomically unstable loci in a sample and/or the quantity of genomically unstable loci, therapeutic treatment can be provided to the patient based on the genomically unstable loci, not only on the presence of a particular type of cancer or location of the cancer in the patient.

A therapeutic treatment is effective when the number of genomically unstable loci decrease. A therapeutic treatment is ineffective when the number of genomically unstable loci either increases or remains the same. The number of genomically unstable loci can be determined by either comparing the number of genomically unstable loci to a reference sequence or to the number of genomically stable loci in the same sample, as described above.

An embodiment of the invention includes a reference log based upon the methods of the invention described above and includes the targeted therapeutic treatments provided to patients based upon the number and/or presence of predetermined genomically unstable loci in a sample and the efficacy of the therapeutic treatments for the patients in treating the cancer.

### Providing Personalized Treatment for Patient Based on Quantitative Assessment

Methods of the present invention include providing a comprehensive diagnosis and treatment to a patient. A sample is obtained from the patient, as described above and a pathology report of the sample is completed. In the report, the pathologist indicates the stage and grade of the cancer in the patient. The information on a comprehensive level, such as tumor size, type of cancer, and tumor location, is considered for treatment purposes.

Genomically unstable loci of the sample are also determined, as described above. Pathology alone is not determinative of the genomically unstable loci associated with the tumor, therefore the specific genomically unstable loci for the sample, as well as the pathology report, are considered in determining the therapeutic treatment for the patient to specifically target the genomically unstable loci of the patient's cancer.

For example, if Patient A presents with Cancer I, the pathology report and treatment suggested would be for Cancer I. Likewise Patient B and Patient C also have Cancer I so under current treatment all three patients would receive the same treatment course. However, further analysis of the samples from Patients A, B, and C show that Patient A has genomically unstable loci AA while Patient B has Cancer I but genomically unstable loci BB, and Patient C also has Cancer I but genomically unstable loci CC. Methods of the present invention provide that Patient A receives therapeutic treatment X, Patient B receives therapeutic treatment Y, and Patient C receives therapeutic treatment Z. Each therapeutic treatment is designed, and known, to specifically target the genomically unstable loci that each patient presents, rather than the cancer alone.

### Qualitative Assessment of Genomically Unstable Loci

Embodiments of the invention provide for assessing cancer in a patient through a qualitative assessment of genomically unstable loci. The qualitative assessment includes identifying the genomically unstable loci and assigning a weighted value to each genomically unstable locus. Methods of the invention provide for identifying genomically unstable loci by the type of genomic instability, the location of the genomic instability, and the amount of genomic material perturbed by the genomic instability. Embodiments further provides for assigning weighted values dependant on the identification step.

Methods of the invention provide for identifying the genomically unstable loci in the sample. As described above, look up tables can be used to compare sequencing results to determine genomically unstable loci of the sequence. Once a genomic sequence from one sample has been determined by sequencing, as described above, hybridization techniques are used to determine variations in sequence between the sample sequence and a reference sequence. The variations between the two sequences are the genomically unstable loci of interest and the type, location, and amount of genetic material affected can also be identified from the variations.

An example of a suitable hybridization technique involves the use of DNA chips (oligonucleotide arrays), for example, those available from Affymetrix, Inc. Santa Clara, Calif. Reference sequences for use in comparison to the sample sequence include, but are not limited to, a sample from a non-cancerous tissue taken from the subject, such as a buccal swab, or a human consensus sequence.

In other embodiments of the invention, a primer with predetermined genomically unstable loci that binds to the nucleic acid of the sample is indicative of that genomically unstable locus. The presence of specific genomically unstable loci in particular cancers can also determine the grade, stage, and prognosis of the cancer. One method of determining the presence of predetermined genomically unstable loci includes PCR. Methods for implementing PCR are well-known. In the present invention, human DNA fragments are amplified using human-specific primers. Amplicon of greater than about 200 bp produced by PCR represents a positive screen. Other amplification reactions and modifications of PCR, such as ligase chain reaction, reverse-phase PCR, Q-PCR, and others may be used to produce detectable levels of amplicon. Amplicon may be detected by coupling to a reporter (e.g. fluorescence, radioisotopes, and the like), by sequencing, by gel electrophoresis, by mass spectrometry, or by any other means known in the art, as long as the length, weight, or other characteristic of the amplicons identifies them by size.

After determining the presence and identity of genomically unstable loci, methods of the invention provide for assigning a weighted value to each genomic instability based on the type, location, and amount of genetic material affect. Methods of the invention further provide for qualitatively assessing the entire sample with a weighted sum. In such an embodiment, the genomic instabilities are characterized by type, location, or amount of nucleotides affected and each category is assigned a weighted value. A weighted sum is then derived by multiplying each category's weighted value times the number of genomically unstable loci within the category. A weighted average may further be calculated by dividing the weighted sum by total amount of genomically unstable loci in each category.

In embodiments of the invention, the weighted value may be any integer or indentifier based on the significance and severity of the genomically unstable locus. The weighted value acts as a means to scale and score genomically unstable loci in comparison to a normal reference sequence and cancer references. Certain embodiments provide for comparing the sequence to a suspected cancer reference sequence in order to scale the sample sequence in comparison to known instabilities found in the suspected cancer sequence. The invention embodies any method of scoring or scaling. In a non-limiting embodiment the weighted value for instabilities may be on a scale from −10 to +10. The +10 may indicate the genomically unstable locus is extremely unstable because its an exact match to instabilities found in highly progressed or developed cancers. An +4 may represent a genomically unstable locus that is latent instability, meaning it will not cause cancer on its own, but may become cancerous upon influence of external factors such as aging and smoking. Whereas +2 may represent a genomically unstable locus found in some undeveloped cancers but nothing directly relates the locus to cancer progression. A zero on the scale may include instabilities not yet known to have any effect or any negative effect towards cancer. A −10 include genomically unstable locus shown not to affect cancers, for example the instability relates to learning disabilities. Further, embodiments provide for the weighted scale to include a +1 for locus the same as those in cancer, 0.5 for locus similar to those found in cancer, and 0 for locus without a causal link to cancers.

In certain embodiments, methods of the invention assign a weighted value based upon the type of genomically unstable locus. The main types of genomic instabilities include subtle sequence changes, alterations in chromosome number, translocations of chromosomes, and single nucleotide polymorphisms. It is recognized that genomic instabilities are linked to cancer, specifically genomic instabilities that lead to accumulation of cell death and cell growth. Several articles expand on the types and characteristics of genomic instabilities leading to cancer including Lengauer, Christoph, et. al. “Genetic Instabilities in Human Cancers.” Nature 396 (1998): 643-49; Shen, Zhiyuan. “Genomic Instability and Cancer: An Introduction.” Journal of Molecular Cell Biology (2011).

Genomic subtle sequence changes include additions, deletions, inversions, and substitutions of one or more nucleotides within a sequence, but not to the extent of large chromosomal sequence changes. A single nucleotide polymorphism (SNP) is a type of genomic subtle sequence change that occurs when a single nucleotide replaces another within the sequence. Alterations in chromosome numbers include additions, deletions, inversions and substitutions of chromosomes within a sequence. Chromosome translocation occurs when a segment of a chromosome attaches, or fuses, to another chromosome, or when noncontiguous segments within a single chromosome fuse. The result of chromosome translocation is the fusion of two different genes, in which the fused genes may have cancer causing properties or the translocation results in the disruption or deregulation of normal gene function. Gene amplifications results when multiple copies of a chromosomal segment are reproduced, instead of a single copy.

After identifying the type of genomically unstable locus, methods of the invention provide for assigning a weighted value to each genomically unstable locus. In certain embodiments, if an addition, deletion, substitution, translocation, inversion, amplification, or single nucleotide polymorphism found in the sample is similar or identical to the same type of instability in a cancer, then a weighted value reflecting its significance and severity will be assigned according. For example, consider a nucleic acid sequence with a genomically unstable locus representing an addition X, genomically unstable locus representing a translocation Y, and a genomically unstable locus representing a SNP. Both the addition, the SNP and the translocation are assigned a weighted value. If the addition X in the sample is exactly the same as an addition X found in cancer X, then addition X will receive a high value, such as +10. If the translocation Y is not yet identified as an exact translocation found in cancer sequences, but is very similar to a translocation Z found in a particular cancer, then the value of the instability will be high, such as a +6, but not as high as addition X, which represented an exact match. If the SNP Y is not found in cancers, then its weighted value may be a 0, or if the SNP Y is identified as a harmless SNP then its weighted value will be −8. The assigned values are aggregated to arrive at a score that can be used to predict grade, stage, and prognosis of cancer

Other embodiments assign a weighted value based upon the location of the genomically unstable locus. In one embodiment, the weighted value is assigned based upon determining on which chromosome the unstable locus resides. Different chromosomes have varying functions. Instabilities in certain chromosomes lead to cancer whereas instabilities in other chromosomes have no link to cancer. Therefore, genomic instabilities on a certain chromosome are often indicative of a certain type of cancer, whereas genomic instabilities on other chromosomes have no link to cancer. For example, genomic instabilities associated with chromosome 14 are linked to leukemia. Csinady, et al., Leukemia 23 (2009): 870-76. Genomic instabilities associated with chromosome 10 are linked to brain cancer. Yadav Et Al., JAMA 302.3 (2009): 276-89. Genomic instabilities associated with chromosome 9 are linked to bladder cancer and brain cancer. Schneider, et al., Cancer Genetics and Cytogenetics 191.2 (2009): 90-96 and Simoneau, Marys, Oncogene 19.54 (2000) 6317-323. Genomic instabilities associated with chromosome 4 are not linked to any cancers and are commonly linked to other genetic diseases, such as Huntington and Parkinson. Bernstam, Victor. Handbook of Gene Level Diagnostics in Clinical Practice. CRC, 1992.

An example of assigning weighted values to genomically unstable loci based upon on which chromosome the loci reside is shown here. Consider a sample in which twenty genomic instabilities are found on chromosome 14, five genomic instabilities are found on chromosome 10, and three genomic instabilities are found on each of chromosomes 4 and 9. Using a scale of −10 to +10 for weighted values, genomic instabilities found on chromosome 14 are assigned a value of +10 because chromosome 14 is highly associated with a cancer and in this sample the chromosome had the highest number of genomic instabilities. Genomic instabilities found on chromosome 4 are assigned a value of −10, because instabilities found on chromosome 4 are not generally associated with cancer. Genomic instabilities found on chromosome 9 are assigned a weighted value of 3 because chromosome 9 is associated with a cancer, however, there are only three genomic instabilities on chromosome 9 as compared to chromosome 14 that has twenty genomic instabilities. Similarly, genomic instabilities found on chromosome 10 are assigned a weighted value of 4 because chromosome 10 is associated with a cancer, however, there are only five genomic instabilities on chromosome 10 as compared to chromosome 14 that has twenty genomic instabilities. Based on different values assigned to each genomic instability, it can be determined that the patient most likely has leukemia and is potentially at risk of developing brain or bladder cancer.

Other embodiments assign a weighted value based upon proximity of the genomic instability to known or suspected locations of instabilities in certain cancer. To carry out such methods, identified genomic instabilities are compared to a specific cancer reference, and then weighed according to their locations in regards to the known instabilities that are associated with the specific cancer. For example, consider Cancer X has a genomically unstable locus in the middle of chromosome A, and another genomically instable locus between chromosomes B and C. A sample has a genomically unstable locus in the middle of chromosome A, and a instability near an end of chromosome B. A high weighted value, such as a +10, will be assigned to the locus in the middle of chromosome A because such locus represents an exact match to location of an instability on chromosome X. The instability near the end of chromosome B will have a lower weighted value because it is not an exact match, however the weighted value should reflect the closeness of the genomic instability near the instability between B and C. For example, if the genomically unstable locus is 2 bases away from the cancer causing instability, its weighted value may be an 7, whereas if the genomically unstable locus is 10 bases away, the weighted value may be a 4. The weighed values may then be used to determine the stage and grade of the sample in relation to Cancer X.

Other embodiments assign a weighted value based upon proximity of the genomic instability to the telomeres. Proximity to telomeres is an important characteristic because telomeres and telomerase are linked to cancer. Telomeres are responsible for regulating cell division by capping chromosomes to prevent the ends of intact chromosomes from appearing like DNA breaks to the DNA replication machinery. Telomeres functioning properly prevent chromosomal degradation, fusion, and rearrangements during DNA replication. With normal cell replication, the telomeres begin to shorten until the telomere is gone and the cell dies. However, in many cancerous cells genomic instabilities may prevent the telomeres from getting shorter by initiating an enzyme called telomerase. Telomerase is found in many cancers and allows mutated cancer cells to replicate indefinitely. The following provide more detailed description of telomeres, telomerase, and genomic instabilities De Lange, T. “Telomere-related Genome Instability in Cancer.” Cold Spring Harb. Symp. Quant. Bio. 70 (2005): 197-204, and Greider, Carol, et al. “Telomeres, Telomerase and Cancer.” Scientific American (2009). Genomic instabilities on or near telomeres may further cause various different fusions, additions, deletions, translocations all of which may contribute to cancer. Therefore, location of instabilities near or on telomeres may provide invaluable insight towards identifying the stage and prognosis of cancer in a sequence.

In determining how to weigh the genomically unstable loci near telomeres, many factors may affect the weighted value such as whether the proximity of the genomic instability to the telomere has been linked to cancer in cancer sequences, the potential of the locus in impacting the telomere's function, type of instability and its proximity to the telomere, the amount of genetic material affected by the instability iii regards to its proximity to the telomere, and the exact location in regards to the telomere, i.e. on the telomere, a base away from the telomere, or a few bases away from the telomere. For example, Cancer X has a genomically unstable locus residing on a telomere. A sample has a genomically unstable locus two bases away from the telomere. Here, the weighted value may be a 8 because two bases is very close to the telomere and such close proximity may have the potential to impact the telomere's function. In another example, consider that genomically unstable loci located on a first telomere of chromosome A are known to be causally linked to Cancer Y and that genomically unstable loci located on the second telomere of chromosome A have not yet been causally associated with Cancer Y. A sample reference has a genomically unstable locus on the first telomere and a genomically unstable locus on the second telomere. The genomically unstable locus on the first telomere will have a 10 because it represents an exact match to Cancer Y. The genomically unstable locus on the second telomere may have a 7, because its telomere is not yet associated with Cancer Y, but telomeres perform similar functions and its location on the same chromosome may result in the instability having the same cancer causing significance.

In certain embodiments, a weighted value is assigned to a genomic instability based upon the amount of genomic material perturbed by the instability, i.e., the number of nucleotides affected by the instability. A weighted value may be assigned based upon the amount of genetic material affected in the aggregate. In this embodiment, weighted values may be assigned to proportionally reflect the amount of material affected in comparison with other locus. For example, an addition affects 4 bases whereas a translocation affects 10 bases. The weighted value for the addition will be 2 whereas the weighted value for the translocation will be 5. The weighted value of 2 for the addition and the weighted value of 5 for the translocation proportionally and comparatively represent the amount of material affected in each locus.

In another embodiment, the amount of genetic material perturbed by a locus or loci may further be characterized by subdividing the amount of genetic material affected into regions. A single genomic instability may be subdivided into regions, or all of the genomic material affected by all of the genomically unstable locus may be placed into regional categories. The regional divisions may include coding v. non-coding and introns v. exons. A weighted value may be assigned to reflect the amount of genetic material affected in each region. In an example, Cancer X has a known genomically unstable locus affecting 10 nucleotides. A nucleic acid from a sample also has a genomic instability at the same genomically unstable locus that is known to be associated with Cancer X, however, the genomic instability from the sample affects only 3 nucleotides. In this case, the sample genomically unstable locus is assigned a value of 3 to reflect the amount of genetic material affected in comparison to the genomically unstable locus associated with Cancer X. In another example, a genomically unstable locus affects 50 bases in a non-coding region and another genomically unstable locus affects 10 bases in a coding region of chromosome Y. The non-coding region may have a value of 2 because non-coding region mutations do not affect protein function. The coding region in the same sample may have a weighted value of 6, even though less bases were affected, because its function in coding protein carries with it a higher cancer causing potential.

In certain embodiments, more than one characteristic of the genomic instability is assessed to determine the value assigned to that instability. For example, an instability can be assigned a value not only based on its type (e.g., addition, deletion, translocation), but also its proximity to a telomere and its proximity to a known cancer causing genomic instability. In one example, the weighted value for a genomically unstable locus represents the severity of the locus factoring in that the locus is an addition (type) and the addition affects multiple nucleotides (amount of genetic material affected). In such an example, the value reflects two characteristics of the locus. In another example, a weighted value represents that the locus is a gene amplification (type) affecting only a small amount of genetic material (amount of genetic material affected) on a certain chromosome (location). Such example factored in all three characteristics in determining the weighted value.

Another aspect of the invention assesses assessing cancer in a patient by analyzing a nucleic acid from a sample, identifying one or more genomically unstable loci in the nucleic acid, categorizing the genomically unstable loci, assigning a weighted value to each category, and assessing cancer based on the weighted values. The categories include but are not limited to the type of genomic instability, the location of the genomic instability, and the amount of genetic material affected. Applying a weighted value to a category reflects the overall influence of the category containing certain genomic characteristics within the sample.

A method of calculating a weighted sum from the weighted values of the categories is provided here. The weighted sum reflects the overall influence of all of the genomically unstable loci within the sample. A weighted sum may be devised by adding each category's weighted value times the corresponding amount of genomically unstable loci in each category or amount of genetic material affected in each category. For example, category 1 has a weighted value of 10 and contains 2 genomically unstable locus and category 2 has a weighted value of 4 and 1 genomically unstable locus. The corresponding weighted sum equals 24, the result of (10×2)+(4×1). The invention further provides for calculating a weighted average where the weighted sum is divided by the amount of genomically unstable locus in the sample. The weighted average may allow for a more manageable value in the case where weighted sums are extremely large. The weighted average using the above weighted sum equals 8 (the weighted value 24/(2 genomically unstable locus+1 genomically unstable locus).

For example, if the genomically unstable categories are based on type, one sample may include a category of deletion, a category of additions, and a category of gene amplifications. A weighted value for each category may be assigned based on the amount of genomically unstable loci in each category. For example, the weighted value is proportional to the amount of loci in the categories. Take a sample that when categorized by type results in two categories, a deletion category with 7 deletion-type genomically unstable loci and an addition category with 3 addition-type genomically unstable loci. Assigning values to the category's proportionally based on amount results in the deletion category having a weighted value of 7 and the addition category of 3. In another embodiment, a weighted value for a category may be the average of the weighted values for each individual genomically unstable locus. The weighted value for each individual genomically unstable locus is assigned based on the above embodiments of the invention. For example, after categorizing, a sample has a deletion category composed of 2 deletions, deletion A was assigned 8 and deletion B was assigned a 4. The resulting weighted value of the deletion category is 6, calculated by adding the weighted values (8+4=12) divided by the number of weighted values (2).

### Providing and Recording Targeted Therapeutic Treatment Based on Quantitative and Qualitative Assessment

Methods of this invention are useful because the size of the tumor may shrink over the course of treatment while the tumor cells may remain as genomically unstable, if not more so, than when the tumor was a larger size. Alternatively, a tumor may remain the same size in an individual, while the weighted values and, or number of genomically unstable loci may decrease, thus decreasing the stage, grade, and prognosis of cancer in the patient. Therefore, by assessing the presence of specific genomically unstable loci in a sample and/or the quantity of genomically unstable loci, therapeutic treatment can be provided to the patient based on the genomically unstable loci, not only on the presence of a particular type of cancer or location of the cancer in the patient.

An embodiment of the invention includes a reference log based upon the methods of the invention described above and includes the targeted therapeutic treatments provided to patients based upon the number and weighted values of genomically unstable loci in a sample and the efficacy of the therapeutic treatments for the patients in treating the cancer. The reference log contains a total assessment of the genomically unstable loci as compared to a reference sequence and sequences of certain cancers. In certain embodiments, the reference sequence is a normal sequence and the cancer sequence is from a cancer the patient is suspected of having, but the sample may be compared to one or more cancer reference sequences for diagnosis purposes.

After the genomically unstable loci in a first sample are identified, quantified and assigned weighted values and sums based on selected characteristics and categories, the quantity of genomically unstable loci and calculated weighted values and sums are recorded in a reference log for the patient. A second sample from the same patient is taken after a lapse in time, during a course of treatment, or after a course of treatment. Methods of the invention are performed on the second sample to identify, quantify, and assign weighted values and sums to the genomically unstable loci using the same scaling methods and the same characteristics and categories used for sample 1.

The second sample's quantity of genomically unstable loci, weighted values and sums are likewise recorded in the patient's reference log. Variances in the quantity and corresponding weighted values and sums between the two samples represents changes in the stage or grade of the cancer. If the second sample is taken after a course of treatment, the variances among the quantity, weighted values, and sums are indicative of whether the course of treatment is effective. Because the weighted values represent each genomically unstable locus, either individually or categorically, the course of treatment can be specifically tailored to treat genomically unstable loci that are not responding to the treatment. For comprehensive diagnostic treatment methods, the reference log may be used in conjunction with a pathology report. Methods of the invention provide for continuing the method of future samples from the patient after diagnosis and over the course of treatment in order to qualitatively assess the progression or regression of the cancer and to determine an appropriate course of treatment.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes which come within the meaning and range of equivalency of the claims are therefore intended to be embraced therein.

1. A method of assessing chromosomal instabilities in a subject, the method comprising:
obtaining genetic material from an effluent, lavage, or organ wash of the subject; and
analyzing the genetic material for a chromosomal instability.

2. The method according to claim 1, wherein prior to the analyzing step, the method further comprises purifying the genetic material.

3. The method according to claim 1, wherein the analyzing step comprises sequencing the genetic material.

4. The method according to claim 1, wherein the analyzing step comprises:
identifying one or more genomically unstable loci in the genetic material; and
assigning a weighted value to each genomically unstable locus.

5. The method according to claim 4, wherein the weighted value is based on a severity of the genomically unstable locus.

6. The method according to claim 4, wherein the weighted value is based on a type of genomic instability.

7. The method according to claim 6, wherein the type of genomic instability is selected from the group consisting of additions, deletions, substitutions, translocations, alterations, amplifications, and single nucleotide polymorphisms.

8. The method according to claim 4, wherein the weighted value is based on a location of the genomically unstable locus.

9. The method according to claim 8, wherein the location is selected from the group consisting of: location on a chromosome, proximity to telomeres, and proximity to known or suspected locations of genomic instabilities found in certain cancers.

10. The method according to claim 4, wherein the identifying step comprises sequencing the genetic material and comparing the sequenced genetic material to a reference sequence to thereby identify the genomically unstable loci.

11. The method according to claim 1, wherein the method is performed again at a later period in time, thereby monitoring progression or recurrence of a disorder associated with the identified chromosomal instability.

12. The method according to claim 1, wherein the sample comprises a tumor cell.

13. The method according to claim 1, wherein the effluent, lavage, or organ wash has previously contacted an organ of the subject selected from the group consisting of lung, colon, bladder, cervix, vagina, kidney, spinal cord, brain, mouth, tongue, throat, and skin.

14. The method according to claim 13, wherein the effluent, lavage, or organ wash is a colon effluent administered during a colonoscopy.

15. The method according to claim 13, wherein the effluent, lavage, or organ wash is a bladder wash administered during a cystoscopy.

16. The method according to claim 13, wherein the effluent, lavage, or organ wash is a cervical wash administered during a cervical exam or pap smear.

17. The method according to claim 1, wherein the genetic material comprises a chromosome or a portion of a chromosome.

18. A method of sequencing a whole genome of a subject, the method comprising:
obtaining a genome from an effluent, lavage, or organ wash of the subject; and
sequencing the genome.

19. The method according to claim 18, wherein prior to the sequencing step, the method further comprises purifying the genome.

20. The method according to claim 18, further comprising identifying loci of the genome that are indicative of a disorder or an abnormality.

21. The method according to claim 20, wherein the method is performed again at a later period in time, thereby monitoring progression or recurrence of a disorder associated with the identified loci.

22. The method according to claim 18, wherein the sample comprises a tumor cell.

23. The method according to claim 18, wherein the effluent, lavage, or organ wash has previously contacted an organ of the subject selected from the group consisting of lung, colon, bladder, cervix, vagina, kidney, spinal cord, brain, mouth, tongue, throat, and skin.

24. The method according to claim 23, wherein the effluent, lavage, or organ wash is a colon effluent administered during a colonoscopy.

25. The method according to claim 23, wherein the effluent, lavage, or organ wash is a bladder wash administered during a cystoscopy.

26. The method according to claim 23, wherein the effluent, lavage, or organ wash is a cervical wash administered during a cervical exam or pap smear.
